(19) United States
(12) Patent Application Publication
(10) Pub. No.: US 2009/0214671 A1
(43) Pub. Date: Aug. 27, 2009

(54) Personalizing Cancer Chemotherapy Based on Methylation and Germ-Line Mutational Analysis of BRCA-1

(76) Inventor: Sven Olek, Berlin (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/297,641

(22) PCT Filed: Apr. 24, 2007
(86) PCT No.: PCT/EP2007/003597

§ 371 Date: Mar. 17, 2009

(30) Foreign Application Priority Data
Apr. 24, 2006 (EP) 06075948.7

(51) Int. Cl. C12Q 1/68; A61K 33/24; A61K 31/337; A61P 35/00

(52) U.S. Cl. 424/649; 435/6; 514/449

(57) ABSTRACT

The present invention relates to a method for personalized diagnosing, prognosing, and treating of diseases, such as cancer, and in particular to a method for the personalized treatment of breast and/or ovarian cancer, based on a methylation and germ-line mutational analysis of the gene BRCA-1.

The present invention relates to a method for personalized diagnosing, prognosing, and treating of diseases, such as cancer, and in particular to a method for the personalized treatment of breast and/or ovarian cancer, based on a methylation and germ-line mutational analysis of the gene BRCA-1.

An estimated 182,000 new cases of invasive breast cancer occurred in the United States during 1995 and over 46,000 deaths resulted from this disease. The search for and identification of specific genetic elements which contribute to the development of breast cancer is an essential part of achieving better treatment and earlier diagnosis.

The discovery of BRCA-1 is a recent example of a burgeoning effort in molecular biology which is focused on the identification of specific disease-associated genes. BRCA-1 is the first gene discovered in an intensive worldwide search for genes associated with enhanced susceptibility to breast and ovarian cancer.

The BRCA-1 gene consists of 100 Kb of DNA which comprises more than 20 coding exons and encodes a protein of 1863 amino acids. (Gene Bank Accession No. U14680). Sequence analysis has provided little insight into BRCA-1 function since only a short region within the amino terminus (comprising less than 10% of the coding sequence) shows significant homology to known protein sequences. Specifically, this region consists of a putative zinc finger domain which may be critical in facilitating interactions between BRCA-1 and other proteins. Although the role this gene plays in breast cancer development is unknown, it is clear that germ-line mutations within this gene are associated with an 87% and 44% lifetime risk for breast cancer and ovarian cancer, respectively, whereas the general female population has a 12% lifetime risk. The BRCA1 and BRCA2 gene mutations are more often identified in breast cancer patients with poor prognostic factors (e.g., estrogen-receptor-negative tumors, higher growth rates, age less than 35 at onset of disease, breast cancer in both breasts).

For BRCA associated tumors, it is assumed that the alleles of BRCA1 and BRCA2 are inactivated before tumor development occurs. BRCA1 and BRCA2 are believed to take part in a common pathway involved in maintenance of genomic integrity in cells; however, little is known about the specific molecular mechanisms involved in BRCA mutation associated (BRCA-linked) ovarian carcinogenesis. For example, it is not known whether BRCA1 and BRCA2 mutations affect common or unique molecular pathways in ovarian cancer, or if these pathways overlap with those involved in the formation of sporadic tumors. Both BRCA proteins have been implicated in important cellular functions, including embryonic development, DNA damage repair, and transcriptional regulation (see Scully and Livingston, Nature 408:429-432, 2000; Zheng et al., Oncogene 19:6159-6175, 2000; Welsh et al., Trends. Genet. 16:69-74, 2000; and MacLachlan et al., J. Biol. Chem. 275:2777-2785, 2000). BRCA1 and BRCA2 have each been implicated in defective homologous recombination DNA repair (see Arvanitis et al., International Journal of Molecular Medicine 10:55-63, 2002), and it is believed that each may be a positive regulator of homologous recombination, with BRCA2 potentially interacting with Rad51, a central homologous recombination effector protein, and BRCA1 regulating GADD45, a DNA damage response gene.

Ovarian cancer has a relative high mortality rate compared to other cancers, due in part to the difficulty of diagnosis. As far as gynecological malignancies are concerned epithelial ovarian cancer is the leading cause of death (see Welsh et al., PNAS 98: 1176-1181, 2001). Studies indicate that the five-year survival rates for ovarian cancer are as follows: Stage 1 (93%), Stage II (70%), Stage III (37%), and Stage 1V (25%) (see Holschneider and Berek, Sermin. Surg. Oncol. 19: 3-10, 2000). Thus, there is a particular need for improved methods for early diagnosis, prognosis, monitoring and treating of ovarian cancer.

Protein and mRNA levels, and changes in these levels, may be associated with specific types of cancer, and cancer progression. Such association is often specific to the type of cancer, which means that what is over-expressed in one cancer may be under-expressed (or unchanged) in another. Thus, a collection or set of genes/proteins that are differentially regulated in a specific cancer may be indicative and specifically diagnostic of that type of cancer.

The molecular mechanisms that are involved in the onset and progression of ovarian cancer remain poorly understood. However, some mutations causing ovarian cancer have been identified. Between 5% and 10% of all ovarian cancers are hereditary. As far as BRCA-1 is concerned, it appears that the percentage of BRCA mutation associated tumors is significantly higher for ovarian cancer than for breast cancer: With a life time risk of approximately 87% for developing breast cancer, a life time risk of app.44% for ovarian cancer, and an approximately 10× higher incidence of breast than ovarian cancer, the proportion of BRCA-1 hereditary tumors appears 5 times higher for ovarian cancer than breast cancer.

While an association of BRCA with breast and ovarian cancers is undisputed, the effect of the different origins of the disease (i.e., spontaneous and/or multifactorial or BRCA-associated) on diagnosis and initial therapy is unclear. After an initial dispute about the effect of the differing disease origins, there seems to be growing consent that—possibly in contrast to the situation observed for breast cancer patients—BRCA associated tumors are associated with better survival prognosis than the spontaneous disease in ovarian cancer. Initially, this clinical observation has been brought in context with the earlier disease onset in inherited cases. However, various studies show that longer survival is also observed independently of age, at least in Ashkenasi Jew populations, with the founder mutations 185_{del}AG and 5382ᵢₙₛC in BRCA1 and 6174_{del}T in BRCA2 (Ben David Y, et al. J Clin Oncol. Effect of BRCA mutations on the length of survival in epithelial ovarian cancers. 2002 Jan. 15; 20(2):463-6. Rubin, S. et al. Clinical and Pathological Features of Ovarian Cancer in Women with Germ-Line Mutations of BRCA1 NEJM Nov. 7, 1997, Lewine D et al J Clin Oncol, Fallopian Tube and Primary Peritoneal Carcinomas Associated With BRCA Mutations, 2003 November, 4222-7). In summary, the data suggest that BRCA associated ovarian cancer patients have a more favorable survival prediction than the average patient. On the other hand, Chiang et al. (Chiang J W, Karlan B Y, Cass L, Baldwin R L. BRCA1 promoter methylation predicts adverse ovarian cancer prognosis. Gynecol Oncol. 2006 June; 101 (3):403-10. Epub 2005 Dec. 19.) describe a comparison of the clinical outcome of ovarian cancer in patients whose tumors contain BRCA1 genes that are silenced by promoter hypermethylation with patients with germ-line BRCA1 mutations, and with patients with wild-type BRCA genes.

The median disease-free interval and median overall survival were significantly shorter for patients with a methylated BRCA-1 promoter (9.8 months) than for BRCA-1 mutation carriers suggesting that methylation of the BRCA-1 promoter is associated with poor outcome. BRCA1 is proposed as part of a global panel of methylated genes associated with aggressive disease.

Olopade and Wei (in: FANCF methylation contributes to chemoselectivity in ovarian cancer. Cancer Cell. 2003 May; 3(5):417-20.) describe a model of ovarian cancer tumor progression implicates aberrant FANCF promoter methylation that is associated with gene silencing and disruption of the Fanconi-anemia-BRCA pathway. Disruption of the pathway occurs de novo in ovarian cancers and may contribute to selective sensitivity to platinum salts. Similarly, D'Andrea (in D'Andrea A D. The Fanconi Anemia/BRCA signaling pathway: disruption in cisplatin-sensitive ovarian cancers. Cell Cycle. 2003 July-August; 2(4):290-2.) describes that ovarian tumors often exhibit chromosome instability and hypersensitivity to the chemotherapeutic agent cisplatin. This cellular phenotype may result from an acquired disruption of the Fanconi Anemia/BRCA from methylation and silencing of one of the FA genes (FANCF), leading to cisplatin sensitivity. The serial inactivation and reactivation of the FA/BRCA pathway is described as having important implications for the diagnosis and treatment of ovarian cancers and related cancers. Both these publications describe FANCF methylation. Thus, not only the platinum-sensitivity should show differences, but also the response to alkylating agents such as Melphalan should be changed depending on the availability of the DNA repair enzymes.

In ovarian and breast cancer, platinum-resistance has been identified as one of the reasons for treatment failure and, consequently, the poor general prognosis. Nevertheless, the opportunity to develop improved, personalized chemotherapies based on a methylation analysis of BRCA-1 has apparently not been recognized. The present invention satisfies this need and provides a variety of related advantages as well.

This object of the present invention is solved by providing a method for determining a patients' response to a chemotherapy for a tumor, comprising a) determining the amount of methylation of the gene for BRCA-1, and b) determining germ-line mutations of the gene for BRCA-1, wherein an increase in the methylation is indicative for a lack of response of said patient to said treatment, if no germ-line mutations of the gene for BRCA-1 are detected.

The present invention is based on the following findings:

a) In non-small cell lung cancer, patients with lower amounts of BRCA-mRNA expression are less likely to develop platinum-resistance.
b) The FANCF gene is functionally located upstream of BRCA-1 controlling BRCA dependent DNA repair. In ovarian and breast cancer, patients with methylation changes in the promoter of this gene are posed with an increased risk of platinum-related treatment resistance.
c) Methylation changes in the BRCA-1 promoter result in shorter overall survival, compared with patients with wild-type BRCA-1 without methylation change. An even more pronounced survival benefit is observed when patients with methylation changes are compared to patients without them, but with BRCA-1 germ-line DNA-mutations.
d) Further studies relating to ovarian cancer prognosis, ostensibly unrelated to platinum-related treatment, suggest longer survival for BRCA-1 mutated patients versus spontaneous cancers.
e) Early disease onset is frequently associated with familial causes and coincides with better prognosis.

Jointly, the findings summarized in points a) and d) assisted by e) are in agreement with the assumption that the inability of BRCA-1 associated DNA repair coincides with the tumors' inability of developing platinum-treatment resistance as a cause for improved prognosis.

In contrast, the findings as mentioned in b) and c), above, explain an increased occurrence of resistance in tumors associated with methylation: When platinum-salts are used for treatment of methylation-associated tumors, selective pressure favors revertants to the unmethylated original, i.e., tumor cells with functional DNA repair mechanisms. While selective pressure is equivalent for germ-line DNA mutations, those occur at a rate approximately 1000-fold less-frequent than methylation changes. Therefore, reverting germ-line mutated cells to platinum-salt resistance is comparably unlikely.

With novel IV/IP-therapy concepts, including higher local platinum-doses, showing success in the general patient population, a personalized treatment depending on the BRCA-related (epi)genotypes can further improve the treatment: Patients with germ-line BRCA-1 mutations receive intensified platinum-based therapy with low risk for developing resistances. Patients with the known methylation-related tumors, and consequently high risk for resistances, might receive platinum with increased local concentrations within individual treatment cycles, but cycles possibly alternating between platinum/paclitaxel, and those omitting platinum.

Preferred is a method according to the present invention, wherein the tumor is selected from breast and/or ovarian tumors and/or metastases thereof. The present invention is particularly useful in those cases where breast cancer has been identified at a progressed stage of the disease.

Further preferred is a method according to the present invention, further comprising a methylation analysis of additional genes, in particular the genes for the Fancomi Anaemia Pathway, in particular FANCF. In addition to the genes for BRCA1, MSH2, BRCA2 and MSH6, and their respective regulatory pathways, in particular the gene MLH 1 can be included into the analysis in order to further improve the diagnosis. Here, usually first genetic mutations are tested, and after a negative result the potential hypermethylation of the promoter of the gene(s) is/are analyzed.

Preferred is a method according to the present invention, wherein said method is performed prior and/or during the chemotherapy and/or before an adjuvant therapy. Chemotherapy for ovarian cancer is most often given through a vein into the bloodstream. Chemotherapy is mostly offered after surgery, if the cancer is Stage 1c or higher. The chemotherapy drugs are usually injected into one of the veins (given ‘intravenously’ or IV) so that they can circulate through the blood stream, for example every 3 to 4 weeks. The treatments are usually repeated 6 times, but sometimes treatments are given up to 12 times. The drugs can be injected over about 3 hours, or they may be given over 24 hours.

All current adjuvant standard treatments include a platinum-based chemotherapy drug. This will either be cisplatin or carboplatin. Both these drugs have been found to be very effective against ovarian cancer. In addition, in a combinatorial treatment plan, a taxol derivative is included as secondary component. However, in January 2003 NICE (the National Institute for Health and Clinical Excellence) revised its earlier recommendation on taxol. NICE now recommend that women should have a choice of treatment with either Paclitaxel (Taxol) and a platinum drug or platinum drugs alone after surgery for ovarian cancer.

For recurrent ovarian cancer other chemotherapy drugs are used, in particular so if clinical recurrence is confirmed less than six months after completing the initial chemotherapeutic treatment. These current recurrences are commonly regarded as derived from platinum resistances In May 2005, NICE updated their recommended treatment options for recurrent ovarian cancer. While in cases of late recurrence (i.e., later than 6 months) more platinum drug treatment, again in combination with paclitaxel (Taxol) is often employed, which is often preceded by a second surgical procedure, in cases of early recurrences taxol on its own is given or liposomal doxorubicin (Caelyx or Doxil) or topotecan (Hycamtin).

A particular problem is the fact that platinum resistance is not generally predictable, and thus patients often receive in the adjuvant setting platinum drugs, only leading to early recurrences. In case there were predictive parameters, foreseeing occurrence of platinum resistance, alternative treatments could be employed even in the earlier stages, i.e., in the adjuvant setting.

Another aspect of the present invention relates to a method for predicting or monitoring a response to a chemotherapy for a tumor according to the present invention, comprising a method according to the present invention in the subject following administration of said chemotherapy. A change of the methylation of BRCA-1 (either increase or decrease) is indicative for a response and/or likelihood of a response of said patient to said treatment. No changes or a decrease of the methylation pattern usually indicate an effect of the therapy as chosen. Monitoring or predicting can also be combined with other methods, such as, for example, CA125 blood tests and/or CT scans or ultrasound scans that are known in the art.

A “gene” in the context of the present invention is meant to include all regions of the chromosome that are involved in the coding and regulation of the marker under analysis, such as the promoter, exon, and intron regions, 5′UTRs and 3′-UTRs, and regulatory elements for the marker found upstream or downstream, such as enhancers or silencers. Preferred are promoter methylation and exon and intron methylation analyses.

The terms “germ-line mutations”, and “SNPs” are interchangeably used in the present specification, and shall mean sequence differences in the genes/markers of interest as described herein that are not the result of the DNA-methylation of said genes (for example as found after bisulfite conversion of the DNA).

Preferred is a method according to the present invention, wherein determining the amount of methylation comprises determining promoter methylation, exon methylation, intron methylation, overall methylation, CpG island analysis, and/or analysis at specific methylation sites. Examples for methylation sites in BRCA1 are described in, for example, Catteau A, Harris W H, Xu C F, Solomon E. Methylation of the BRCA1 promoter region in sporadic breast and ovarian cancer: correlation with disease characteristics. Oncogene. 1999 Mar. 18; 18(11):1957-65.

Further preferred is a method according to the present invention, wherein determining the amount of methylation comprises a method selected from hybridization, bisulfite conversion, restriction analysis, PCR, rtPCR, sequencing, and/or primer extension. These methods are all well known in the state of the art.

Still further preferred is a method according to the present invention, wherein one or more germ-line mutations are determined in the gene for BRCA-1. Since the breast cancer susceptibility gene brca1 was isolated (Miki et al., 1994), more than 300 disruptive germ-line mutations within the coding region of the gene have been identified in cases of familial breast and ovarian cancer (Couch F J, Weber B L. Mutations and polymorphisms in the familial early-onset breast cancer (BRCA1) gene. Hum Mutat. 1996; 8(1):8-18.) Couch et al., 1996).

Diagnostic sequencing of genes for DNA-repair is generally known in human genetics (Higuchi M, Wong C, Kochhan L, Olek K, Aronis S, Kasper C K, Kazazian H H Jr, Antonarakis S E. Characterization of mutations in the factor VIII gene by direct sequencing of amplified genomic DNA. Genomics. 1990 January; 6(1):65-71), and is offered in specialized centers for the more common indication of breast cancer. These analyses are reasonable and required, since they allow for the confirmation of mutation-related preventive measures. Due to the markedly lower general incidence of ovarian cancer and due to the lack of efficient preventive measures, in ovarian cancer there are no general measures to include mutational tests and a follow up with current molecular markers such as CA125 in families with higher risks. Ii is a preferred embodiment of this invention to couple regular CA 125 tests as a consequence of a positive BRCA mutation test.

The term “associated with” means to include an increased risk of developing the disease. Independently thereof, both the diagnostic analysis of the germ-line mutations in both BRCA genes itself and their importance as a marker for the predisposition can be regarded as fully established and accepted. For the MLH1-gene that is associated with familial colon cancer (and ovarian cancer), diagnostic sequencing is also established. Nevertheless, mutations are commonly indirectly detected using the analysis of micro-satellite instability.

Another aspect of the present invention relates to a method for determining the risk of a patient to develop a chemotherapy-treatment resistant tumor, comprising a method according to the present invention, and determining the risk of a patient to develop a chemotherapy treatment resistant tumor, based on said determinations, wherein an elevated and/or increase in the methylation of BRCA-1 is indicative for an increase risk of said patient to develop a chemotherapy-treatment resistant tumor, if no germ-line mutations of the gene for BRCA-1 are detected.

Additionally provided herein are methods for the classification of ovarian tumors as chemotherapy resistant tumors based on the analysis according to the present invention. Using the data as obtained, multiple types of comparisons can be made to provide qualitative and quantitative information about the tumor-type. Non-limiting examples of such comparisons include visual examination of color profiles of hierarchically clustered markers on a cDNA microarray, multidimensional scaling to the determine relative distance of the analyzed markers, and compound covariate prediction analysis to statistically classify a given tumor into one of two classes, e.g. chemotherapy resistant tumors or non-resistant (sensitive) tumors. In a specific non-limiting example, methylation ratios are generated and used in order to classify tumor types.

Treating a disease includes inhibiting or preventing the partial or full development or progression of a disease (e.g., ovarian cancer and/or breast cancer), for example in a person who is known to have a predisposition to a disease. An example of a person with a known predisposition is someone having a history of breast or ovarian cancer in his or her family, or who has been exposed to factors that predispose the subject to a condition, such as exposure to radiation. Furthermore, treating a disease refers to a therapeutic intervention that ameliorates at least one sign or symptom of a disease or pathological condition, or interferes with a pathophysiological process, after the disease or pathological condition has begun to develop. By way of example, a treatment can be selected from chemotherapy, radiotherapy, or surgical removal of the affected tissue and/or surrounding area, and combinations of the given treatment options.

Another aspect of the present invention relates to a method for diagnosing or prognosing development or progression of cancer in a subject, comprising a method according to present invention, and diagnosing or prognosing development or progression of said cancer based on said determinations.

The results of the comparisons as above can also be used to diagnose or provide a prognosis of progression of ovarian cancer in a subject. The patterns of expression can also be used to screen for therapeutic agents for the treatment of ovarian cancer, or monitoring response to therapy in a subject, by looking for a return of the patterns of expression of the ovarian tumor toward a non-tumor tissue pattern.

Provided herein are furthermore methods of diagnosing or prognosing development or progression of ovarian cancer in a subject, which methods involve detecting altered methylation of BRCA 1 (“marker” or “gene of interest”). In certain embodiments, altered expression is detected in more than marker, for instance in FANCF, MSH2, BRCA2, MLH1, and MSH6 (“markers” or “genes of interest”), and their respective regulatory pathways. In fact what we try is to predict a risk as observed from mutations (or methylation changes) in either one of the named genes. While, mutations in one of the genes predict better survival upon platinum based treatment, no mutations and in particular methylation changes, when found in BRCA 1, FANCF, or BRCA 2, predict worse outcome.

Another aspect of the present invention then relates to the use of platinum salts, cisplatin, and/or paclitaxel for the production of a medicament for the chemotherapy of tumors exhibiting an increased methylation of the gene BRCA 1 in a patient, wherein said chemotherapy is performed using increased local concentrations of platinum salts, cisplatin, and/or paclitaxel within individual treatment cycles, wherein the treatment cycles preferably alter between platinum and paclitaxel cycles and cycles omitting platinum, wherein the tumor is selected from breast and/or ovarian tumors and/or metastases thereof.

Kits are also provided for performing the analyses and diagnoses as above, and the kit may include components for performing a method according to the present invention. Preferred is a kit according to the present invention, comprising an oligonucleotide capable of measuring methylation using established methodologies, such as restriction enzymes, bisulfite conversion followed by RT-PCR, MS-SnuPE and others as well as novel methods to detect methylated parts of the sequence of the gene for BRCA-1, optionally together with oligonucleotides for FANCF, MSH2, BRCA2, MLH1 and/or MSH6. Even more preferred is a kit comprising a solid-surface, preferably a nucleic acid “chip”, for performing methylation and SNP analysis of the markers as described simultaneously together on said chip, onto which the respective oligonucleotides as required are immobilized.

Also encompassed are methods for ovarian cancer therapy, in which a method according to the present invention as above helps in the selection of a treatment regimen. In some examples, the treatment selected is specific and tailored for the subject, based on the analysis of that subject's profile for one or more ovarian cancer-related methylation markers according to the present invention.

Further embodiments are methods of screening for a compound useful in treating, reducing, or preventing ovarian cancer or development or progression of ovarian cancer. Such methods involve determining if a test compound alters the methylation profile of a subject (or cells of an in vitro assay), and selecting a compound that so alters the methylation profile. In specific examples of such methods, the test compound is applied to a test cell. Also encompassed are compounds selected using the methods described herein, which are useful in treating, reducing, or preventing ovarian cancer or development or progression of ovarian and/or breast cancer. Such compounds can be formulated into a pharmaceutical preparation, in particular a medicament.

While for cases where BRCA germ-line mutations are found, it might not be essential to test the methylation status, it is, however, essential to test for such mutations, if a methylation change has been found for the BRCA gene. If the methylation change is found to be concomitant with point mutations, this is associated with better prognosis, and—as a consequence—with a recommendation for platinum therapy.

Ovarian carcinoma (OC) are often diagnosed at a progressed stage of the disease. A standard therapy of the OC optimally consists of a removal of the malign tissue, with the aim of a macroscopic removal of most of the tumor. Surgery in general is followed by 6 cycles of an intravenous taxol/carboplatin therapy. The overall time of survival has not been improved for a long time, and the overall prognosis—with about 20% long-term survival—remains disillusioning. Nevertheless, some important successes in the understanding of the molecular basis of the disease have been achieved in the context of the present invention, which should lead to better concepts of treatment.

One of the reasons for the ovarian carcinoma are defects in several DNA-repair enzymes. As basis for familial forms of the breast and ovarian cancer, amongst others, mutations in the genes BRCA-1 and BRCA-2 are known. In addition, there are indications that the genes MHL1 and MSH2 that are responsible for an inherited form of colon cancer (HNPCC), are also associated with an increased risk to develop ovarian cancer.

Specifically in the context with BRCA-1, a dramatic consequence of the different mechanistic background was shown. The median progression-free free survival of carriers of the genetic changes is found at 39.5 months (overall survival: 78.6 months) compared with 9.5 months (35.6 months) with carriers of epigenetic changes.

This connection as described between the basis of the defect and a clinically very different prognosis can be explained by the fact that with standard platinum-therapy a strong selection pressure is exerted onto the cells. Since platinum as strong complexing agent modifies the DNA, the ability of the cell to develop platinum resistances is tightly connected with the ability for DNA repair. If the DNA repair enzyme encoding genes are defect, the platinum-therapy remains effective. A re-mutation that re-establishes the repair function in the inherited forms of the disease is extremely unlikely, and correspondingly rare. In contrast to this, the reversal of an epigenetically induced inactivation as is observed in sporadic cancers, is not unlikely, and has even to be awaited under strong selection pressure. The thus achieved ability for a repair increases the chance of survival of the malign cells, thus can explain both the generation of platinum resistances as well as the different clinical prognosis.

Platinum medicaments are considered the most effective anti-cancer substances of all and in particular in ovarian cancer show high response rates (platinum hypersensitivities). Nevertheless, the treatment often results in the development of resistances. Accordingly, platinum resistances account for the biggest difficulties that block a curative OC-treatment, and prevent an improvement of the survival prognosis. In order to avoid such resistances, and to improve the overall survival, in adjuvant chemotherapy different experimental strategies are pursued:

a) Optimization of the taxan-platinum-combination and their bioavailability, e.g. by intraperitoneal therapies (Armstrong D K, Bundy B, Wenzel L, et al. (2006). Intraperitoneal cisplatin and paclitaxel in ovarian cancer. N Engl J Med 354 (1): 34-43),
b) Increase of the density of dosages (e.g. high-dose chemotherapy or decrease of intervals) (Frickhofen N, Berdel W E, Opri F, Haas R, Schneeweiss A, Sandherr M, Kuhn W, Hossfeld D K, Thomssen C, Heimpel H, Kreienberg R, Hinke A, Mobus V; Phase 1/11 trial of multicycle highdose chemotherapy with peripheral blood stem cell support for treatment of advanced ovarian cancer. Bone Marrow Transplant. 2006 October; 38(7):493-9), and
c) Inclusion of a third substance into the taxan-carboplatin-combination, so-called triplets (e.g. addition of Epirubicin, Topotecan or Gemcitabine) (du Bois A., Weber B., Pfisterer J. Epirubicin/Paclitaxel/Carboplatin (TEC) vs. Paclitaxel/Carboplatin (TC) in First-Line Treatment of Ovarian Cancer FIGO Stages I to IV. Interim Results of an AGO-GINECO Intergroup Phase 111 Trial Meeting. ASCO Annual Meeting 2001; Abstract 805.).

High-dose chemotherapy and a more precise local administration of the substances within one of the therapeutic concepts as known exhibit improved response rates (Armstrong D K, et al, above). In contrast to this, the assignment of triplets with the main focus of overcoming platinum resistances did not result in improved profiles of survival (du Bois A., et al., see above).

The reasons for the different success of the above concepts could be explained as follows: It has to be assumed that the term “adenocarcinoma-type ovarian carcinoma” relates to a series of very diverse disease patterns. The different molecular biological bases and their influence on a response to the therapeutics in recent studies remain mostly ignored. Thus, without a diagnostic differentiation, it is a prerequisite for a recognition of the success of a therapy that in the overall population (i.e. in all OC-subtypes) a significant advantage for survival is achieved. Nevertheless, this can only be observed if mechanisms are triggered that are relevant for all or the majority of the OC-subtypes. An example for this is the intraperitoneal therapy that allows for a better bioavailability and higher dosages, and thus offers an equivalent advantage for all patient groups (Armstrong D K, et al, see above). After the general effectivity of the platinum derivatives and taxan compounds has been optimized over the years additional dramatic improvements of the overall survival based on a further improvement of these methods do not appear promising.

As long as the patient groups can not be diagnostically differentiated, the search for drugs is particularly problematic, if a benefit for survival is only given for a selection of patients, such as, for example, those with platinum resistant tumors. Here, reaching a statistic significance becomes more difficult, the more specific the combination of medicaments is targeted towards individual subtypes. That is: the less patients gain a profit, the more difficult it becomes to recognize this in the overall group. Thus, no dramatic improvements of the therapies are awaited with the diagnostic and prognostic methods as currently used. Thus, new approaches are mandatory required.

In the context of this invention, a method was established that allows for a recognition of the molecular reason of the platinum resistance in advance. Thus, the risk for a resistance shall be qualified, and, starting from this, the adjuvant treatment shall be defined. The early recognition of potential resistances is of high importance for the overall treatment concept. Currently, resistances are recognized during the progression of treatment through the progression of the tumor growth during or shortly after chemotherapy. Since in principle only adjuvant therapies can lead to a cure, this finding can not be used any more in the sense of a curative therapy that could lead to successive clinical improvements.

In the treatment of ovarian cancer in particular information with respect to the patient specific reaction towards platinum medicaments are of importance, since, in principle alternative medicaments are already available. These medicaments—such as, for example, Topotecan, Epirubicin, 5-Fluorouracil, Gemcitabine, or hexamethyl melanin as classical cytostatics, or modern medicaments—such as Tarceva, Iressa, Avastin or Erbitux—find their uses in substitution therapies, where platinum medicaments can be used, such as, for example at relapse therapies with obvious platinum resistances. Today, these rather efficient drugs are generally not employed in the “first-line” therapy, since then the tendency towards a platinum resistances must be known in advance. Without such knowledge the platinum therapy still is regarded as the standard for all patients. This is problematic, since relapse patients are principally subjected to a palliative therapy, whereas patients after optimal surgery and in the adjuvant disease state can be curatively treated (after optimal surgery and effectiveness of the platinum therapy, the chance for a cure increases to more than 50%). Thus, the non-recognition of probable platinum resistance before the first chemotherapy for these patients means the loss of any possibility for a cure, if the platinum therapy fails due to platinum specific resistances, and thus relapses are generated. In contrast, in cases of an early recognition, an alternative therapy would markedly improve the prognosis for survival and, optionally, cure some of the most problematic cases. The principal value of a categorization into resistant und sensitive patients that was already shown in relapse patients (Markman M, Bundy B N, Alberts D S, Fowler J M, Clark-Pearson D L, Carson L F, Wadler S, Sickel J. Phase 111 trial of standard-dose intravenous cisplatin plus paclitaxel versus moderately high-dose carboplatin followed by intravenous paclitaxel and intraperitoneal cisplatin in small volume stage 111 ovarian carcinoma: an intergroup study of the Gynecologic Oncology Group, Southwestern Oncology Group, and Eastern Cooperative Oncology Group. J Clin Oncol. 2001 Feb. 15; 19(4): 1001-1007. Markman M, Markman J R, Zanotti K M, et al Duration of response to second-line platinum based chemotherapy for ovarian cancer: Implications for patient management and clinical trial design. Proc Am Soc Clin Oncol 2003; 22 Abstract 1795: 447.), is not based on molecular results, but on the experience that patients with early relapses often react worse to platinum in the second chemotherapy. Patients with platinum sensitive tumors, that is, longer disease-free interval, often profit from the additional surgeries, and show markedly higher response rates in the platinum chemotherapy (Markman M, et al, above, Lichtenegger W, Sehouli J, Buchmann E, Karajanev C, Weidemann H. Operative results after primary and secondary debulking-operations in advanced ovarian cancer (AOC) J Obstet Gynaecol Res. 1998 December; 24(6):447-51. Eisenkop S M, Spirtos N M. Procedures Required to Accomplish Complete Cytoreduction of Ovarian Cancer: Is there a Correlation with Biological Aggressiveness and Survival? Gynecologic Oncology 2001; 82: 435-441).

U.S. Pat. No. 6,773,897 describes a related strategy, but with a reverse logic as suggested herein, that a hypermethylated promoter of a DNA repair gene leads to an inactivation of the respective enzyme. In consequence, chemotherapeutic agents work better once the DNA repair gene is methylated, since there is no “defensive” action anymore by the natural DNA repair mechanisms. The logic in the present application is opposite to the one above: the present inventors found that a methylation in the promoter, while causing disease, leads to a phenotype that is adverse to a successful chemotherapeutic treatment when compared to germ-line mutations in the DNA of BRCA, since a methylation of a gene promoter must be reinstated after each cell division. Cancer cells, with their fast division rate, are therefore extremely likely to loose the “wrong” methylation phenotype, and therefore reactivate the BRCA repair mechanism. Therefore, the inventors differentially determine the methylation pattern in BRCA and DNA repair related cascades and their primary DNA sequences, in order to predict adverse (methylation phenotype) or good (DNA mutation rate) response(s) to chemotherapeutic measures/treatments.

In a preferred embodiment of the method according to the present invention, the following decisions based on possible analytical outcomes are made for therapy:
BRCA germ-line mutation and an aberrant methylation leads to a good response. Therefore, a treatment would be recommended as present/initiated, with platinum-based first line.
BRCA germ-line mutation and no aberrant methylation leads to a good outcome, again no change of the standard therapy would be reasonable.

Nevertheless, in both cases as above, in a second line treatment physicians could possibly be encouraged to consider platinum-based therapies, even if clinical recurrence occurs early (i.e., before 6 months after the last cycle of 1st line treatment).
No BRCA germ-line mutation and no aberrant methylation phenotype. In this case, no better indication is given, and therefore, no individualized treatment recommendation can be given.
No BRCA germ-line mutation, but an aberrant methylation phenotype. This phenotype has a particular bad prognosis, possibly due to the mechanism explained above. Recommendation for treatment would in this case tend to only cautiously or not use platinum-based first line treatments. If platinum is unavoidable, the recommendation would possibly be to use no cis- or carboplatinum, but newer derivatives, such as oxaliplatin, which has been suggested to be able to overcome standard platinum resistant tumors. Alternatively, other approved first line treatments include Taxol® Paclitaxel, Alkeran®, Melphalan, Adriamycin®, and Rubex®-Doxorubicin.

One preferred embodiment of the invention is the use of genetic and epigenetic information in the BRCA1, BRCA2, RAD51, FANCD2, FANCG, FANCE, FANCF, MLH1, MSH2, and MSH6 genes, and combinations thereof with BRCA1 for the treatment and outcome-prediction in ovarian and breast cancer.

A further preferred embodiment is to measure the given parameters for outcome prediction on chemically converted, such as by sodium bisulfite-treated, DNA.

A further preferred embodiment of the present invention is, to measure the genetic and epigenetic effects in parallel on a single platform, which—despite the chemical conversion—allows for the detection of all potential genetic mutations with the exception of 24 mutations that display CG to TG mutations as well as changes of epigenetic changes in the named DNA fragments in one reaction and detection mode.

The invention shall now be described further in the following examples with respect to the accompanying drawings, without being limited thereto. For the purposes of the present invention, all references as cited herein are incorporated by reference in their entireties. In the figures,

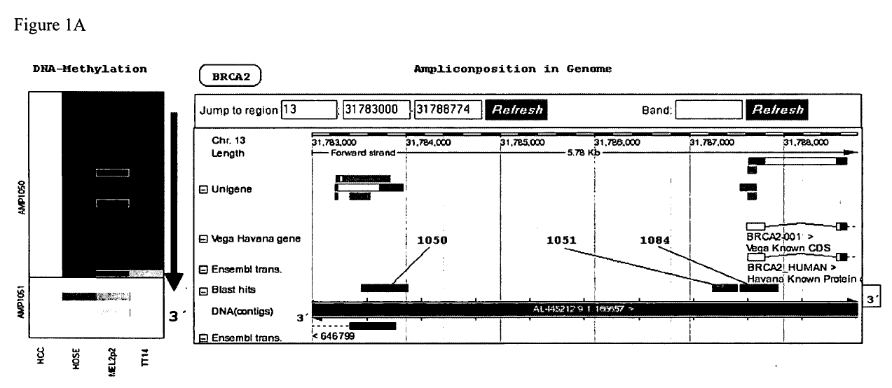

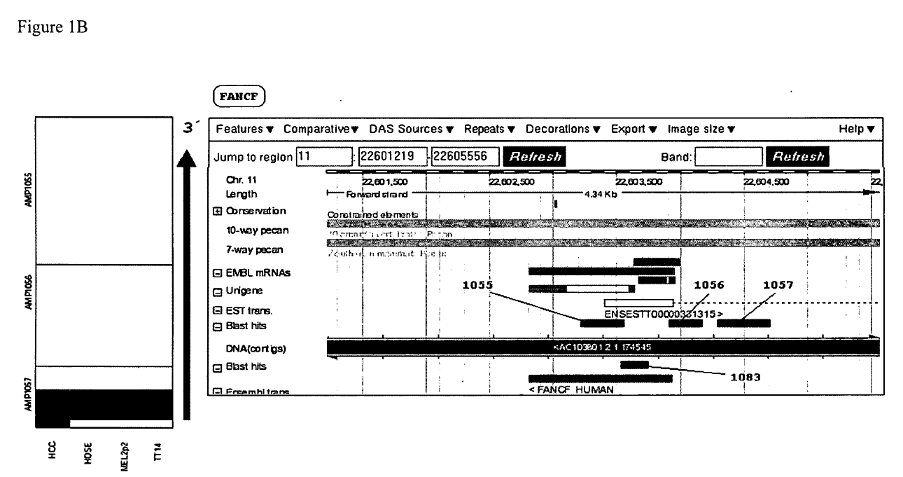

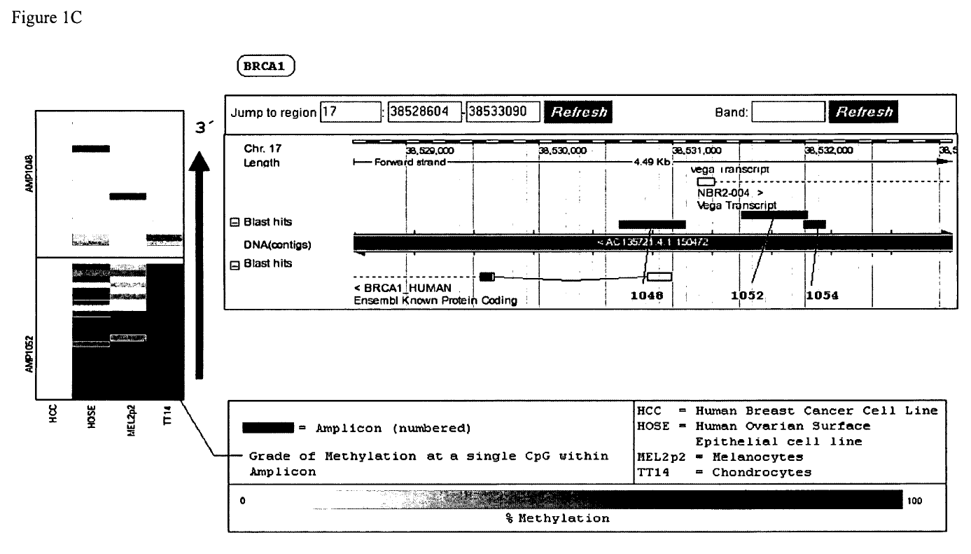

FIGS. 1A to 1C show in the left panel the methylation pattern in the selected preferred gene regions (BRCA2, FANCF and BRCA1) tested in four different samples including different healthy primary cell types (TT14 and Mel2p2) as well as a human breast cancer cell line and a human ovarian cancer cell line (HOSE).

### EXAMPLES

Methylation determination using bisulfite-based sequencing was performed in the following encoding and gene promoter regions: BRCA1, BRCA2, RAD51, FANCD2, FANCG, FANCE, FANCF, MLH1, and MSH2.

In this example, the inventors analyzed genetic regions and genes in ovarian cancer cell lines, breast cancer cell lines, and healthy primary cells derived from different germ-layers. This analysis was designed to find appropriate amplicons and gene regions with respect to two aspects: First, it was important to find amplicons that are specifically amplified in the bisulfite converted sequence. Second, the inventors were interested in finding regions that were sensitive to differential methylation in healthy tissues. The latter experiment should give indications as to which regions are suitable or excellent candidates for aberrant methylation signals in diseased tissues.

In order to allow for a parallel measurement of methylation and mutation in the indicated genes, it is important to compare the original sequence with the bisulfite converted sequence including all genetic mutations that changed from C, G and A to T. Specifically mutations from C to T become unrecognizable in the bisulfite treated version as they could be derived either from a mutation (germ-line mutation or SNP) or from chemical treatment. In order to circumvent this difficulty, methylation has to be measured on the negative original and converted strand, since there the mutation result in an “A” whereas the original unchanged base would become a “G”. Therefore, there is no loss of information in those regions where methylation is measured on either of the two bisulfite-treated strands derived from the negative original strand, whereas in all other mutations, the bisulfite-treated versions of the original positive strand is measured or—for mutations derived from G to A—vice versa (in the positive strand) when the original negative strand is chosen for the main template.

For the table of the current example as given below, mutational information was taken from the mutation information database at www.genome.gov, an information network provided by the National Human Genome Research Institute. The database provides detailed information about genetic mutations in the BRCA1 gene.

In the present example, the reference as used is from the BRCA1 sequence, accessible on GenBank at accession number HSU14680. It is intended to use enhanced information as derived from other databases or extensions of the current ones as well as mutations in other DNA repair genes for the generation of the sequences according to the present invention. The given mutated positions are compared with positions where base changes from C to T are found during the bisulfitation process. The comparison works as follows: every T in the converted sequence is identified, and the original is labeled based on the genomic sequence position.

The inventors then analyze the different possibilities that could be responsible for the base change in the bisulfite-treated sequence, which are, on one hand, “T”s from genetic mutations (germ-line mutations or SNPs) in the original sequence, or, on the other hand, base changes exclusively caused by the bisulfite treatment process—which are therefore of no biological significance.

At positions where C to T mutations have been reported in the original sequence database, the negative strand of the bisulfite-treated (and original) sequence is used for SNP detection. From information derived from this strand, relevant mutations can be extracted. Below, table 1 shows an example for those positions that include changes in the original sequence that become a “T”, and are therefore lost in bisulfite strand 1. In the context of the method of the present invention, opposite strand oligonucleotides are designed for these sequences, in order to still be capable to detect genetic mutations. Envisaged is also the opposite situation, i.e., where the negative original strand is used in order to detect the majority of SNPs, and only a selection of positions is analyzed on the positive strand.

TABLE 1
Mutations displaying changes from C, G and
A to T in the original sequence of BRCA1.

| Exon | NT | Codon base-change | AA change |
|---|---|---|---|
| 11 | 1068 | 317 C to T | Gln to Stop |
| 11 | 1113 | 332 C to T | Arg to Trp |
| 11 | 1115 | 332 G to T | Arg to Arg |
| 11 | 1120 | 334 C to T | Pro to Leu |
| 11 | 1152 | 345 G to T | Asp to Tyr |
| 11 | 1155 | 346 C to T | Pro to Ser |
| 11 | 1164 | 349 G to T | Glu to Stop |
| 11 | 1173 | 352 G to T | Glu to Stop |
| 11 | 1185 | 356 C to T | Gln to Stop |
| 2 | 122 | 1 G to T | Met to Ile |
| 11 | 1221 | 368 G to T | Glu to Stop |
| 11 | 1240 | 374 C to T | Thr to Ile |
| 11 | 1260 | 381 A to T | Lys to Stop |
| 11 | 1327 | 403 C to T | Ser to Phe |
| 11 | 1371 | 418 G to T | Glu to Stop |
| 11 | 1377 | 420 G to T | Asp to Tyr |
| 2 | 138 | 7 C to T | Arg to Cys |
| 11 | 1452 | 445 G to T | Glu to Stop |
| 11 | 1515 | 466 C to T | Arg to Trp |
| 11 | 1518 | 467 A to T | Lys to Stop |
| 2 | 153 | 12 C to T | Gln to Stop |
| 11 | 1537 | 473 A to T | Asn to Ile |
| 11 | 1569 | 484 G to T | Gly to Stop |
| 11 | 1590 | 491 C to T | Gln to Stop |
| 11 | 1599 | 494 C to T | Gln to Stop |
| 11 | 1605 | 496 C to T | Arg to Cys |
| 11 | 1629 | 504 C to T | Arg to Cys |
| 11 | 1639 | 507 G to T | Arg to Ile |
| 11 | 1653 | 512 C to T | Leu to Phe |
| 11 | 1680 | 521 GC to TA | Ala to Stop |
| 11 | 1690 | 524 C to T | Ala to Val |
| 11 | 1695 | 526 C to T | Gln to Stop |
| 11 | 1731 | 538 C to T | Gln to Stop |
| 11 | 1735 | 539 C to T | Thr to Met |
| 11 | 1740 | 541 C to T | Gln to Stop |
| 11 | 1749 | 544 C to T | Gln to Stop |
| 11 | 1795 | 559 G to T | Gly to Val |
| 11 | 1806 | 563 C to T | Gln to Stop |
| 11 | 1822 | 568 C to T | Pro to Leu |
| 11 | 1866 | 583 A to T | Lys to Stop |
| 11 | 1875 | 586 C to T | Pro to Ser |
| 11 | 1908 | 597 G to T | Glu to Stop |
| 11 | 1938 | 607 A to T | Lys to Stop |
| 2 | 194 | 25 C to T | Pro to Ser |
| 11 | 1959 | 614 A to T | Lys to Stop |
| 11 | 1984 | 622 C to T | Ala to Val |
| 11 | 1985 | 622 G to T | Ala to Ala |
| 11 | 1989 | 624 G to T | Glu to Stop |
| 11 | 2016 | 633 C to T | Pro to Ser |
| 11 | 2019 | 634 C to T | Pro to Ser |
| 11 | 2031 | 638 G to T | Glu to Stop |

Of particular important are those sequences, where C changes to T (bold). For those positions, the negative strand is used for a production of oligonucleotides for the detection of SNPs.

1. A method for determining a patients' response to a chemotherapy for a tumor, comprising:
a) determining the amount of methylation of the gene for BRCA-1, and
b) determining germ-line mutations of the gene for BRCA-1, wherein an increase in the methylation is indicative for a lack of response of said patient to said treatment, if no germ-line mutations of the gene for BRCA-1 are detected.

2. The method according to claim 1, wherein the tumor is selected from breast and/or ovarian tumors and/or metastases thereof.

3. The method according to claim 1, further comprising a methylation analysis of one or more genes for the Fancomi Anaemia Pathway.

4. The method according to claim 1, wherein said method is performed prior to, and/or during, the chemotherapy and/or before an adjuvant therapy.

5. The method according to claim 1, wherein determining the amount of methylation comprises determining promoter methylation, exon methylation, intron methylation, overall methylation, CpG island analysis, and/or analysis at specific methylation sites.

6. The method according to claim 1, wherein determining the amount of methylation comprises at least one method selected from hybridization, bisulfite conversion, restriction analysis, PCR, rtPCR, sequencing, and primer extension.

7. The method according to claim 1, wherein said chemotherapy is a therapy based on platinum salts, cisplatin, and/or paclitaxel.

8. The method according to claim 1, wherein one or more germ-line mutations are determined in the gene for BRCA-1.

9. The method according to claim 1, wherein said method further comprises determining the risk of the patient to develop a chemotherapy-treatment resistant tumor based on said determinations.

10. A method for monitoring and/or predicting a response to a chemotherapy for a tumor, comprising a method according to claim 1, and further comprising monitoring the methylation of the gene BRCA-1 in the patient following administration of said chemotherapy, wherein a change of the methylation pattern of the gene BRCA-1 in the patient is indicative and/or predictive for a response of said patient to said chemotherapy.

11. A method for diagnosing or prognosing development or progression of cancer in a patient, comprising a method according to claim 1, and further comprising diagnosing or prognosing development or progression of said cancer based on said determinations.

12. A chemotherapy method comprising administering platinum salts, cisplatin, and/or paclitaxel to a tumor exhibiting an increased methylation of the gene BRCA-1 in a patient, wherein said chemotherapy is performed using increased local concentrations of platinum salts, cispiatin, and/or paclitaxel within individual treatment cycles, and wherein the tumor is selected from breast and/or ovarian tumors and/or metastases thereof.

13. A kit comprising components or materials for performing a method according to claim 1.

14. The kit of claim 13, comprising an oligonucleotide capable of hybridizing to the nucleic acid of a methylation related part of the sequence of the gene for BRCA-1.

15. The kit of claim 13, comprising a nucleic acid chip for performing methylation and germ-line mutational analysis simultaneously on said chip.

16. The method, according to claim 12, wherein the treatment cycles alter between platinum and paclitaxel cycles and cycles omitting platinum.

17. The method, according to claim 3, comprising a methylation analysis of one or more genes selected from FANCF, MSH2, BRCA2, MLH1 and MSH6.
